(19) United States
(12) Patent Application Publication
(10) Pub. No.: US 2008/0312169 A1
(43) Pub. Date: Dec. 18, 2008

(54) Cosmetic use of D-ribose

(76) Inventors: Clarence Albert Johnson, Wyoming, MN (US); John A. St. Cyr, Coon Rapids, MN (US)

Correspondence Address: Kathleen R. Terry, 13840 Johnson Street NE, Ham Lake, MN, 55304, US

(21) Appl. No.: 11/725,893

(22) Filed: Mar. 20, 2007

(51) Int. Cl. A61K 31/7105; A61P 17/00

(52) U.S. Cl. 514/44

(57) ABSTRACT

D-ribose is incorporated into a cosmetic composition and applied to the skin to reduce the length and area of wrinkles and to improve the complexion of the skin.

### BACKGROUND OF THE INVENTION

The skin is the interface between an organism and the external environment. As such, it is continually subject to stresses such as extreme heat or cold, attack by microorganisms, exposure to UV radiation, abrasion, chemical irritants and the like. As a result, the skin shows signs of 15 damage such as sunburn, roughening, wrinkling, discoloration, and even malignancies, including basal cell carcinoma, squamous cell carcinoma and melanoma. While these effects are often considered to be normal aging, in fact, they are not normal results of aging but are responses to damage.

Many of the effects may be related to free radical formation or suboptimal cell function. The skin is an organ with a high metabolic rate and a high cell turnover. Because it serves as a barrier to microbes, chemicals, radiation, heat and water, skin is highly impermeable. The dermis or corium layer contains blood vessels and nerves. It projects into the epidermis or outer layer in ridges called papillae. The cells of the dermis secrete a ground substance and collagen that give support to the epidermis. The dermis also contains hair follicles and oil glands.

The epidermis is composed of five layers. The basal layer cells are columnar and arranged perpendicularly. These cells divide rapidly to provide a continually renewing epidermis. The basal layer is also the site of melanin formation. As the layers are pushed up towards the surface of the skin by the formation of new cells, they become progressively more dehydrated and keratinized to form the stratum spinosum, flattened polyhedral cells with short processes or spines; the stratum granulosa, composed of flattened granular cells; the stratum lucidurn, composed of several layers of clear transparent cells in which the nuclei are indistinct or absent, and the stratum corneum or cornified layer. This layer is comprised of flattened, dead, keratinized cells that form a barrier to the external environment. As the final step in the ever-renewing metabolism of the skin, the stratum corneum gradually flakes off. Integrity of the skin require good function in all layers.

In damaged skin, the dermis may secrete less collagen or the collagen may become damaged by free radicals or radiation and lose elasticity, resulting in sagging and wrinkling. UV radiation causes the stimulation of the basal cells to produce protective melanin. While a tanned skin is considered a sign of health, actually, a tan is a response to UV damage which causes damage to the collagen.

Finally, some skin may have an epidermis that is unnaturally dry and flaky, possibly because of sub-optimal turnover of the dermal cells. Besides detriments to health in having a less functional barrier to infection, damaged skin is less aesthetically pleasing. Unfortunately, the exact areas that are most exposed to the environment and sustain the most damage are those that the subject presents to the world, that is, the face and hands.

Many products have been marketed to improve skin function and appearance. A popular preparation is a cream that incorporates alpha-hydroxy acids, which appear to function as mild irritants that stimulate the exfoliation of the most external stratum corneum, thereby exposing the less cornified and less dry-appearing layers. Unfortunately, many subjects find that these acids are too irritating and may actually cause a red, rough rash.

Some efforts have been made to improve the condition of skin by applying nutrients topically, on the theory that these nutrients will penetrate the dermis and speed up the turnover of dermal cells, thus presenting younger, more youthful appearing cells to the surface. An example is U.S. Pat. No. 5,053,230, issued Oct. 1, 1991, in which a nutrient medium previously found to support in vitro culture of human epithelial cells to promote the trophism of the skin and related follicles. This complex mixture contains amino acids, monosaccharides, nucleosides and vitamins. Simpler compositions containing sucrose are on the market.

The need remains for a composition to be applied topically to stimulate the growth of dermal cells, especially fibroblasts, thereby reducing wrinkles.

### SUMMARY OF THE INVENTION

The present Invention concerns the use of D-ribose, in a cosmetic composition designed to optimize the metabolism of skin cells. This new use is especially intended to improve cellular respiration of intracellular ATP so as to optimize the metabolism of cutaneous cells and tissues.

The inventors have measured the in vitro effect of D-ribose on the cellular energetic metabolism by the consumption of oxygen and the concentration of cellular ATP. They have demonstrated that D-ribose increases oxygen consumption, both mitochondrial and cytosolic, and regulates the production of ATP by the cells, improving the metabolism of the cutaneous cells and tissues.

The effect of ribose on the skin has also been evaluated in vivo, by profile measurements measurement of wrinkles and fine wrinkles, by measuring the DPM of the cutaneous elasticity and by self-evaluation of a panel of volunteers.

The present invention has the object of using ribose in a cosmetic composition to optimize the metabolism of skin cells. The invention is more precisely directed to the use of ribose as the sole active ingredient to optimize the metabolism of skin cells. The compositions disclosed herein are intended for topical use. It has been found that ribose, when used as the basis for a cosmetic composition, permits the lessening or prevents the appearance of wrinkles and crow's feet, improves the cutaneous elasticity and improves the brilliance of the skin.

When D-ribose is used, it should be as pure as possible, preferably at least 98% pure as measured by HPLA. In the most preferred composition, D-ribose is incorporated in the body of a cosmetic composition as the sole active agent. According to the invention, D-ribose can be used in various ways: in solution, in a carrier, in microcapsules or in association with a mixture of excipients such as: vegetable or mineral oils, vegetable or mineral waxes, silicone, alcohols or fatty acids, or surface-active agents.

The cosmetic compositions for topical use according to the invention can be formulated in various manners, such as simple emulsions, complex emulsions or microemulsions, aqueous gels, liposomes, oils, sprays, sticks, aerosols, or toilet products such as shampoos or shower gels, These compositions can be used on the face, the body, the scalp and the hair.

D-Ribose can be combined with other ingredients such as: anti-age products, sebaceous regulators, or hydrating compounds. Preferably, the cosmetic compositions of the invention contain from 0.01 to 10%, preferably from 0.1 to 5% by weight D-ribose, and most preferably from 0.1 to 1.5% by weight in respect to the total weight of the composition.

The following examples are given for purposes of illustration of the invention, but are not to be considered as limiting the scope of the appended claims. In particular, these examples use D-ribose obtained by biological fermentation, of a purity greater than 98%.

### DETAILED DESCRIPTION OF THE INVENTION

It is known that ATP plays an essential role in the function of all cells. The level of ATP produced may be too low in respect to the requirements of the cells. ATP molecules are constantly recycled in order to furnish the necessary energy. When skin cells are exposed to different stresses, including pollution, smoking, physical exertion, aging, etc., the cells require more energy than can be recycled. The level of ATP may be decreased, affecting cellular metabolism.

The inventors have observed that incorporation of ribose into a cosmetic composition permitted a significant improvement of cellular respiration and the de novo production of ATP. The inventors measured an in vitro effect of ribose on the cellular energy metabolism, measured by oxygen consumption and intracellular ATP concentration. These in vitro studies demonstrated that ribose acted to augment oxygen consumption as measured by mitochondrial and cytoplasmic respiration and regulated the production of ATP by the cells and would indicate that affect cutaneous cells and tissues. The results of the in vitro studies are shown in Examples 1 and 2.

The following examples are provided to further explain how to make and use the invention, but are not limitations on the scope of the claims appended hereto.

### EXAMPLE 1

### Effect of D-Ribose on the Respiration of Cutaneous Cells In Vitro

The theory that applicants propose is that the application of ribose to fibroblast cells will accelerate the growth and improve the condition of the fibroblast and ultimately the dermis derived therefrom. The culture of fibroblasts is well known. The Coriell Institute for medical research publishes a web site with detailed instructions, the teachings of which are incorporated by reference. (Http:/locus.umdnj.edu/cer/faq/fibro.html.)

The inventors measured the effect of D-ribose on mitochondrial and cytoplasmic respiration of normal human dermal fibroblasts (NHDF) in culture. The cells were cultivated at 37° C. under an atmosphere of 5% CO, in a culture solution of DMEM supplemented with 10% bovine fetal serum.

The NHDF were grown to a monolayer, put in suspension and distributed in the wells of a kit (BD Oxygen Biosensor System The bottom of the well was constituted of a membrane on which is deposited a molecule whose fluorescence is quenched in the presence of oxygen in the incubation medium.

As a measure of oxygen consumption by the cells, the fluorescence will increase as oxygen is depleted.

0.05% D-ribose was added to six wells. The intensity was determined photometrically. The respiratory rate was calculated from the intensity of the fluorescence. It was observed after two hours of contact, that an augmentation of mitochondrial respiration of 31% and of cytoplasmic respiration of 37% over control was seen in the D-ribose supplemented cells. Thus, D-ribose augments the consumption of oxygen in the cells, and consequently, the metabolism of cutaneous cells and tissues.

The inventors have measured the effect of D-ribose on the production of ATP by NHDF in culture, under conditions of hypoxia.

The cells used were cultivated in flasks of normal human epidermal cells (NHDF) in a medium of DMEM with 10% fetal bovine serum, incubated at 37° C. in an atmosphere of 5% CO, and 95% air until 80% confluent. On Day 1, a first measure of the level of ATP was determined on the non-treated, non-stressed NHDF. Two sets of six duplicates were 15 pretreated for 24 hours with 0.05% ribose or not treated as control. At Day 2, before the induction of hypoxia, one set of treated and control cells was harvested to measure the level of ATP. The cells of the second set were suspended, the medium renewed with DMEM with 10% fetal bovine serum with HEPES buffer in order to maintain the pH within physiological conditions during hypoxia and with EDTA in order to prevent the agglutination of the cells. The cells were held in an incubator held at 37° C., in which the atmosphere had been previously replaced by nitrogen. The cells were incubated for six hours in a condition of severe hypoxia (oxygen<2%) in the presence of 0.05% D-ribose versus the non-treated control. Recovery of the cells and measurement of the ATP was followed in the treated and control cells. After six hours of hypoxia, the presence of 0.05% D-ribose permitted recovery of the ATP levels to about 19% of the base levels, showing a partial recovery in these extreme conditions.

### EXAMPLE 3

### Effect of D-Ribose on Cutaneous Elasticity Measured In Vivo

The inventors showed the effect of D-ribose on cutaneous tissue by DTM. Measurement of cutaneous elasticity was determined with the aid of a twistometer before and 28 days after treatment at the area of the cheekbones. A group of 20 women applied a cosmetic product containing 0.5% D-ribose (example 6 of Table 1) twice daily on their entire faces. After 28 days of treatment, a significant variation of the values as compared to Day 0 was observed. The product containing 0.5% D-ribose caused an average 12.3% increase in cutaneous elasticity.

### EXAMPLE 4

### Effect of D-Ribose on Wrinkles Measured In Vivo

The effect of D-ribose on wrinkles was shown by profile measurement. Skin prints of wrinkles were made in the area of crow's feet before treatment, after 14 days of treatment and after 28 days of treatment. A group of 20 women volunteers applied a cosmetic product containing 0.5% D-ribose (example 6 of Table I) twice daily on their entire faces. After 14 days of treatment, a significant variation of the values as compared to Day 0 was observed. The cosmetic product containing 0.5% D-ribose caused an average decrease of 17.6% in the average length of wrinkles, of 18.6% of the total surface area of wrinkles.

### EXAMPLE 5

### Self-Evaluation of the Panelists of the Brilliance of Complexion

A group of 20 women volunteers applied twice daily for 28 days a cosmetic product containing 0.5% D-ribose (example 6 of Table I) on their entire faces. After completing a questionnaire, 67% of the volunteers found that their complexions were more brilliant and more luminous, 71% that their complexions were less dull and more even.

### 6. Representative Compositions.

The following compositions are given as examples of the diverse compositions that can be made, following the teachings of these inventors. Each includes D-ribose as the active ingredient.

These compositions are conveniently and easily applied directly to those areas most in need of improvement, especially the face and hands.

TABLE I
Anti-wrinkle Emulsion for the Face. Ingredients expressed as grams

| | |
|---|---|
| D-Ribose | 0.5 |
| Chelator | 0.10 |
| Emulsifier | 5 |
| Anti-oxidant | 0.05 |
| Isononyl isonanoate | 6 |
| Stearyl heptanoate | 3 |
| Silicone | 6 |
| Preservative | 0.80 |
| Fragrance | 0.25 |

Demineralized water to 100 ml

TABLE II
Oxygenating Mask for the Face

| | |
|---|---|
| D-Ribose | 1% |
| Glycerine | 4 |
| Chelator | 0.10 |
| Emulsifier | 4 |
| Thickening | 0.30 |
| Vitamin B | 0.30 |
| Vegetable oil | 12 |
| Soy isoflavon | 4 |
| Silicone | 6 |
| Anti-microbial agent | 0.80 |
| Fragrance | 0.25 |

Demineralized Water to 100 ml.

TABLE III
Firming Cream for the Body

| | |
|---|---|
| D-Ribose | 1% |
| Glycerine | 2 |
| Chelator | 0.01 |
| Emulsifier | 4 |
| Gelling Agent | 0.3 |
| Vegetable Oil | 3 |
| Vegetable fibre | 2 |
| Silicone | 7.5 |
| Anti-microbial Agent | 4.8 |
| Fragrance | qs |

Demineralized Water to 100 ml

We claim:

1. A method to enhance the metabolism of the skin comprising the topical application of D-ribose.

2. The method of claim 1 wherein the D-ribose is incorporated in a pharmaceutically acceptable carrier at a concentration of 0.01 to 10% weight to volume.

3. The method of claim 1 wherein the D-ribose is incorporated in a pharmaceutically acceptable carrier at a concentration of 0.1 to 1.5% weight to volume.

4. The method of claim 1 wherein the condition of the skin is improved by reducing the length and area of wrinkles and toning the complexion.
